(19) United States
(12) Patent Application Publication
(10) Pub. No.: US 2014/0179646 A1
(43) Pub. Date: Jun. 26, 2014

(54) HORMONE DELIVERY SYSTEM AND METHOD

(71) Applicant: Baucom Institute for Longevity and Life Enhancement, Inc., Overland Park, KS (US)

(72) Inventor: Karan Y. Baucom, Overland Park, KS (US)

(73) Assignee: Baucom Institute for Longevity and Life Enhancement, Inc., Overland Park, KS (US)

(21) Appl. No.: 14/189,801

(22) Filed: Feb. 25, 2014

Related U.S. Application Data
(63) Continuation-in-part of application No. 12/818,798, filed on Jun. 18, 2010, now Pat. No. 8,658,628.
(60) Provisional application No. 61/218,301, filed on Jun. 18, 2009.

(51) Int. Cl. A61K 31/57; A61K 31/565

(52) U.S. Cl. CPC A61K 31/57; A61K 31/565
USPC 514/170

(57) ABSTRACT

A hormone delivery system and method are provided for administering bioidentical human hormones using a combination of modalities for the treatment of human physiological conditions.

### CROSS-REFERENCE TO RELATED APPLICATIONS

This application is a continuation-in-part of and claims priority in U.S. patent application Ser. No. 12/818,798, filed Jun. 18, 2010, now U.S. Pat. No. 8,658,628, issued Feb. 25, 2014, which claims priority in U.S. Provisional Patent Application No. 61/218,301, filed Jun. 18, 2009, both of which are incorporated herein by reference.

### BACKGROUND OF THE INVENTION

1. Field of the Invention

The present disclosed technology relates generally to the administration of human hormones, and in particular to various delivery methods of bioidentical hormone formulations.

2. Description of the Related Art

Hormones are chemical substances produced by cells and organs of the body that affect organs and body systems. Hormones are important for cardiovascular maintenance, musculature, the skeletal system, and cognitive functioning. The body's production of hormones and how the body reacts to hormones affect the well being of individuals and the aforementioned body systems.

The body has three general categories of sex hormones; androgens (ex. testosterone), estrogens (estradiol and estrone), and progestagens (ex. progesterone). Particular female sex hormones and their associated organs are, for example, testosterone (ovaries), estrone and estradiol (ovaries), and progesterone (ovaries and placenta). Testosterone promotes the growth and maintenance of the skeletal system, musculature, and connective tissues, to name a few. Estradiol and estrone principally affect the female reproductive system. Progesterone affects the female menstrual cycle, and maintenance of pregnancy. Hormone deficiencies caused by aging, disease states, exogenous and endogenous environmental conditions, and certain prescribed medications can upset the balance of sex hormones within the body and affect general well being, lifespan, quality of life, and may lead to disease states as well. Therefore, in order to counter the negative effects of hormone deficiencies, patients are often prescribed hormone replacement therapy (HRT) by their treating physicians.

HRT is a system of treatment using either synthetic sex hormones, or bioidentical sex hormones to treat the effects of diminished sexual hormone levels in peri-menopausal, menopausal, and post-menopausal women. Synthetic sex hormones are the predominant type of hormone proscribed in HRT. The types of techniques used in HRT to deliver sex hormones include pills, capsules, gels, creams, patches and troches. Use of synthetic sex hormones in HRT comes with significant problems such as heart problems, cancers and other undesirable side effects. Furthermore, the type and amount of sex hormone administered is limited by dosing regimens associated with the available delivery techniques. Patient selection of desired delivery techniques and source of sex hormones will increase the effectiveness of therapy and compliance. Moreover, the ability of a physician to tailor the amount of sex hormone delivered with each dosing regime will increase the effectiveness of HRT. Therefore, there is a need for a system of HRT that avoids the undesirable side-effects of synthetic hormone treatments, and that provides the patient with a range of techniques for administering the hormones to maximize well being and maintenance of body systems.

Heretofore there has not been available a hormone delivery system with the advantages and features of the disclosed technology.

### SUMMARY OF THE INVENTION

A hormone delivery system is disclosed providing for administration of bioidentical human hormones using a combination of modalities for the treatment of human physiological conditions where treatment by hormone therapy is indicated. Bioidentical estrogen, progesterone and androgen preparations may be administered individually, or in combination to a patient using one or more modalities such as transdermal absorption or ingestion. The hormone delivery system can be used to treat pre-menstrual tension syndrome, peri-menopause, menopause, post-menopause, progesterone deficiency, estrogen dominance, libido issues, and birth control.

When using bioidentical hormones for the efficacy of treating peri-menopausal and menopausal women, it is best to simulate the natural menstrual cycle. Utilizing estrogen throughout the 28 day cycle (from day 1 to day 28), and introducing progesterone as early as day 10 or as late as day 14, can produce these results. During tests, this resulted in producing a period, primarily during the last 5 days of the month. The progesterone portion of the cycle dominates the shedding or secretory phase of the process. The intensity would vary depending on the dosage of estrogen based upon its proliferation of the endometrial lining.

A “steady state” occurs where there is little or no bleeding during the secretory phase. This is based upon the endometrial layer being so scant that the progesterone does not induce bleeding. Previously it was a concern that the endometrial layer would remain and become exposed to the estrogenic component. However, as long as progesterone is also being introduced, there is a ratio that results in an equilibrium wherein the endometrial layer neither grows nor sheds.

Estrogen dominance can occur when large doses of estrogen are predominantly utilized without being balanced out with progesterone. Inducement of the cycle with the use of progesterone with the intact uterus can help to prevent endometrial hyperplasia. Therefore, any female patient utilizing estrogen in a delivery system should also balance that with progesterone.

The type of progesterone used is also important. Oral micronized progesterone and various progestogens are available. Progestogens include natural progesterone and synthetic progestins.

Existing solutions provided by physicians do not incorporate both estrogen and progesterone in a physiological manner that can be simply dosed to the patient. A preferred solution would include a distribution system that is pre-packaged and self-explanatory to the patient.

An embodiment of the present invention presents a hormone delivery system (HDS) which provides the hormones to the patient in a biologically, physiological manner which simulates the menstrual cycle. These hormones can be presented in a number of ways. Transdermal applications include patches, gels, creams, or some combination thereof. The estrogen must be presented in a transdermal application, but the progesterone could also be presented orally, such as in a capsule.

If issues arise, such as breast tenderness and extensive bleeding, this can alert the patient and the treating physician to mechanical causes (e.g. fibroids, endometriosis, or andenomyosis) or that the estrogenic component is being given in such a way that, combined with the patient's own endogenous estrogens, have created estrogen dominance. The remedy is to decrease the estrogenic component of the HDS.

The advantage of the present invention is to provide an HDS that is modifiable by the treating physician by adjusting the dosages. As an example, using gels and creams, the physician can prescribe 25 milligrams per application. The hormones being provided through the HDS are well suited for these variations.

### BRIEF DESCRIPTION OF THE DRAWINGS

The drawings constitute a part of this specification and include exemplary embodiments of the present invention illustrating various objects and features thereof.

FIG. 1 box diagram illustrating the relationship between various elements of an embodiment of the present invention.

FIG. 2 is a chart diagramming a timeline based upon various elements and aspects of the present invention.

### DETAILED DESCRIPTION OF THE PREFERRED EMBODIMENTS

### I. Introduction and Environment

As required, detailed aspects of the disclosed technology are disclosed herein; however, it is to be understood that the disclosed aspects are merely exemplary of the disclosed subject matter, which may be embodied in various forms. Therefore, specific structural and functional details disclosed herein are not to be interpreted as limiting, but merely as a basis for the claims and as a representative basis for teaching one skilled in the art how to variously employ the disclosed technology in virtually any appropriately detailed structure.

### II. Embodiment or Aspect of the Hormone Delivery System

The hormone delivery system embodying the principles of the disclosed technology provides for administration of compositions containing bioidentical human hormones in an amount sufficient to provide therapeutic effect, using a combination of modalities, for the treatment of human physiologic conditions. Examples of specific bioidentical hormones which may be used include, but are not limited to estrogens (estrone, estradiol), progesterone, and androgens (testosterone, androstenedione, dihydrotestosterone (DHT), dehydroepiandrosterone (DHEA)) isomers and derivatives thereof, and combinations thereof Examples of specific modalities which may be used include, but are not limited to topical preparations (gel, cream), ingested articles (tablet, lozenge, capsule, troches), and articles for transdermal absorption of hormone preparations (transdermal patch, impregnated matrices). The type and amount of hormones involved in the various bioidentical human hormone compositions, and the modalities used varies depending independently, or in conjunction with, the physiologic sequence based on the normal menstrual cycle pattern, and specific clinical syndromes involved. A single bioidentical hormone or a combination of bioidentical hormones may be used with any particular modality.

The hormone delivery system may be used for the treatment of conditions related to hormone imbalances or deficiencies where treatment by hormone therapy is indicated. For example, the types of conditions for which the hormone delivery system may be used include, but are not limited to: pre-menstrual tension syndrome; peri-menopause, menopause, post-menopause; progesterone deficiency; estrogen dominance; and libido issues. Additional treatment measures include providing birth control. In conjunction with treatments using estrogens, the hormone delivery system is used in conjunction with indole-3-carbinol, di-indole methane, or flax seed to protect biochemically from hydroxylation of estrogen.

### III. Alternative Embodiment or Aspect of the Hormone Delivery System 2

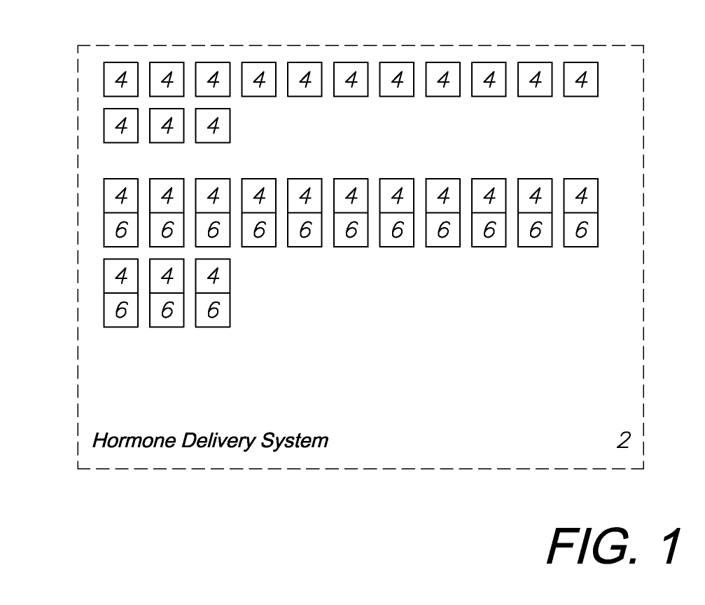

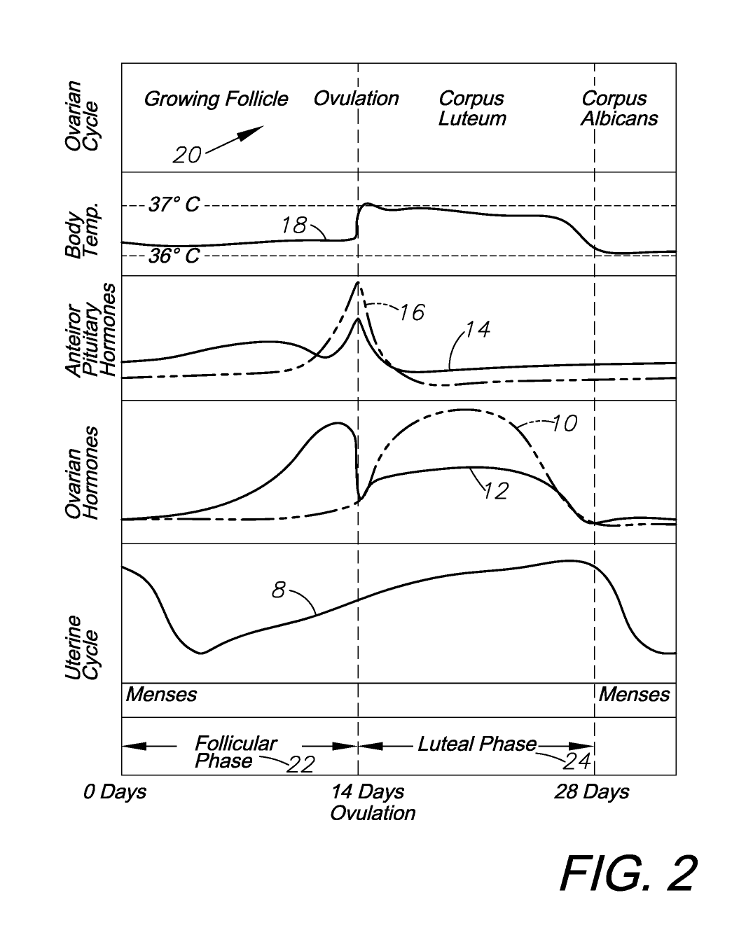

As shown in FIG. 1, a pre-packaged hormone delivery system (HDS) 2 comprising another embodiment or aspect of the disclosed technology can be used for treating peri-menopause, menopause and post-menopause. The bioidentical human hormones estrogens 4 (estrone, estradiol), progesterone 6 using a combination of modalities mentioned above are administered to a patient in a sequential format following the human physiological twenty-eight day menstrual cycle as diagrammed in FIG. 2. Any combination of patches, capsules and other types of delivery systems can be utilized, provided the menstrual cycle is simulated.

Various combinations of estrogen/progesterone can be administered as a cream with suitable proportions and dosage ranges.

The present invention is pre-packaged for clinical efficiency. This ensures the proper dosage without requiring the physician to mix and match the estrogen and the progesterone doses separately. The hormones being applied through the delivery system are FDA approved in a therapeutic fashion for hormone replacement therapy. Examples include 17-B estradiol, progesterone, progestogens, and progestins.

The delivery system is intended for involving estrogen throughout the standard menstrual cycle (from day one through the entire month) and utilizing progesterone as early as day ten or as late is day 14. This ensures progesterone is initiated in addition to the estrogen for the remainder of the cycle. This results in a period primarily during the final 5 days of the month based upon the dosage of estrogen, as it is the estrogen component that proliferates the endometrial lining.

The shedding of secretory phase, as shown in FIG. 2, is under the dominance of the progesterone portion of the cycle, initiating the menstrual cycle. The bleeding can be controlled based upon how much estrogen is provided and as to how the endometrial lining responded in its proliferation.

The present invention simulates the physiological menstrual cycle as its template, mimicking the normal rhythmic delivery of the sex steroids. FIG. 2 illustrates this in some detail. The graph element 8 illustrates the uterine cycle; namely, the thickness of the endometrial lining during a typical cycle. Element 10 graphs the progesterone hormone level over that cycle, and element 12 illustrates the estradiol level. Similarly, element 14 illustrates the follicle-stimulating hormone (FSH) while element 16 represents the lutenizing hormone (LH). Body temperature along the cycle is illustrated at element 18. The phases of the cycle are listed at 20. The follicular phase is represented at 22 and the luteal phase at 24.

Summarizing, the hormone delivery system of this embodiment provides estrogen and progesterone replacements. The estrogen would be provided via a topical solution such as a gel or cream, but the progesterone could be provided either through a micronized oral progesterone (e.g., capsule, sub lingual, troche, or pill) or through a topical, preferably vaginal, gel, cream, or transdermal spray. Dosage ranges for the progesterone can range between 20 milligrams through 300 milligrams.

The 28 day cycle may initiate the progesterone component as early as day 10 or as late as day 14. The component is used cyclically in association with continuous bioidentical estrogen.

By way of example and without limitation, estrogen can be administered as a patch, gel or cream in suitable dosages of, for example, 0.25 mg, 0.375 mg, 0.5 mg, 0.75 mg and 1.0 mg. Progesterone can be administered as a patch, gel, cream, capsule or sublingual in suitable dosages of, for example, 25 mg, 50 mg, 75 mg, 100 mg, 200 mg and 300 mg. Di-indole methane/3-indole carbinol (Dim/3IC) can be administered as 400 mg tablets or gels. However, estrogen-dominant patients should only be given progesterone during days 1-25 of their menstrual cycles. If any estrogen is used, an option is to utilize 3IC/Dim for pathway protection for increasing 2-meo/2 hydroxylation excretion. It is generally important for the patient to excrete estrogen.

Support for the concepts disclosed herein can be found in the following list of references, where are incorporated herein by reference:
Cameron, Sharon T. et al., Continuous Transdermal Oestrogen and Interrupted Progestogen as a Novel Bleed-Free Regimen of Hormone Replacement Therapy for Postmenopausal Women, British Journal of Obstetrics and Gynaecology, October 1997, Vol. 104 pp. 1184-1190.
Campagnoli, C. et al., Progestins and Progesterone in hormone Replacement Therapy and the Risk of Breast Cancer, Journal of Steroid Biochemistry and Molecular Biology, July 2005 Vol. 96 No. 2 pp. 95-108.
Role of Progestogen in Hormone Therapy for Postmenopausal Women: Position Statement of the North American Menopause Society (Retired), Menopause: The Journal of the North American Menopause Society, Vol. 10 No. 2, pp. 113-132.
William Faloon, Surprise Findings in Estrogen Debate, Life Extension Magazine, November 2013.
   IV. Alternative Embodiment Hormone Delivery System for Treatment of Libido issues with Menopause

An alternative embodiment of the present invention seeks to treat libido issues, specifically but not exclusively during menopause, by providing a combination of estrogen, testosterone, and progesterone to the patient. Testosterone will be prescribed to the patient from day 7 through day 20 of the 28 day cycle.

V. Alternative Embodiment Hormone Delivery System for Treatment of PMDD, PMS, and Estrogen Dominance

An alternative embodiment of the present invention uses a combination of estrogen and progesterone at dosages of 25 mg, 50 mg, 100 mg, or 200 mg to treat PMDD, PMS, and/or estrogen dominance. This combination can also be used to treat insomnia in menopausal and post-menopausal women.

VI. Alternative Embodiment Hormone Delivery System for Treatment of Vaginal Dryness, Atrophy, and/or Dyspareunia

An alternative embodiment of the present invention uses a combination of estradiol and testosterone to treat vaginal dryness, atrophy, and/or dyspareunia. A combination of 2 mg/ml of estradiol and 5 mg/ml of testosterone is applied intravaginally 2-3 times per week. This is not to be used for menopausal systemic therapy.

IV. Conclusion

It will be appreciated that the components of the hormone delivery system can be used for various other applications. Moreover, the hormone delivery system can be fabricated in various sizes and from a wide range of suitable materials, using various manufacturing and fabrication techniques.

It is to be understood that while certain aspects of the disclosed technology have been shown and described, the disclosed technology is not limited thereto and encompasses various other embodiments and aspects.

Having thus described the invention, what is claimed as new and desired to be secured by Letters Patent is:

1. A method of delivering hormones to a patient, which method comprises the steps of:
providing a predetermined quantity of human hormones;
determining a hormone deficiency in the patient;
matching a hormone with the determined hormone deficiency;
delivering progesterone and estrogen to the patient in a sequential format following the human physiological twenty-eight day menstrual cycle using a modality comprising transdermal absorption of said estrogen and oral absorption of said progesterone; and
thereby treating hormone imbalances or deficiencies associated with peri-menopause, menopause and post-menopause.

2. The method according to claim 1, wherein said absorption of said progesterone comprises micronized progesterone in a form chosen from the list including: a capsule, a sublingual, a troche, and a pill.

3. The method according to claim 1, wherein said estrogen is delivered to the patient throughout the entire twenty-eight day menstrual cycle.

4. The method according to claim 3, wherein said progesterone is delivered to the patient from day 10 to day 28 of the menstrual cycle.

5. The method according to claim 3, wherein said progesterone is delivered to the patient from day 11 to day 28 of the menstrual cycle.

6. The method according to claim 3, wherein said progesterone is delivered to the patient from day 12 to day 28 of the menstrual cycle.

7. The method according to claim 3, wherein said progesterone is delivered to the patient from day 13 to day 28 of the menstrual cycle.

8. The method according to claim 3, wherein said progesterone is delivered to the patient from day 14 to day 28 of the menstrual cycle.

9. The method according to claim 1, wherein said progesterone is provided at an amount between 20 mg and 300 mg per dose.

10. The method according to claim 1, wherein said estrogen is provided at an amount between 0.25 mg and 1.0 mg per dose.
